(19) United States
(12) Patent Application Publication
(10) Pub. No.: US 2016/0287431 A1
(43) Pub. Date: Oct. 6, 2016

(54) Systems and Methods for Transferring Heat with a Portion of a Mammal

(71) Applicant: AVAcore Technologies, Inc., Ann Arbor, MI (US)

(72) Inventor: Charles A. Hixson, Jr., Ann Arbor, MI (US)

(73) Assignee: AVAcore Technologies, Inc., Ann Arbor, MI (US)

(21) Appl. No.: 15/038,697

(22) PCT Filed: Nov. 18, 2014
(86) PCT No.: PCT/US2014/066144

§ 371 Date: May 23, 2016

Related U.S. Application Data
(60) Provisional application No. 61/907,127, filed on Nov. 21, 2013.
(60) Provisional application No. 61/993,719, filed on May 15, 2014.

(51) Int. Cl. A61F 7/02; A01K 29/00

(52) U.S. Cl. CPC A61F 7/02; A01K 29/00; A61F 2007/0036

(57) ABSTRACT

Systems, kits and methods for transferring heat with a portion of a mammal are provided. Aspects of the subject systems include a negative pressure device configured to apply negative pressure to a portion of a mammal, and a temperature modulation device configured to contact the portion of the mammal under negative pressure in a manner sufficient to transfer heat with the contacted portion of the mammal, e.g., either remove heat or introduce heat into the contacted portion of the mammal. The disclosed systems and methods find use in a variety of different applications, including both therapeutic and non-therapeutic applications.

### CROSS-REFERENCE TO RELATED APPLICATIONS

Pursuant to 35 U.S.C. §119 (e), this application claims priority to the filing date of U.S. Provisional Patent Application Ser. No. 61/907,127 filed Nov. 21, 2013 and to the filing date of U.S. Provisional Application Ser. No. 61/993,719 filed May 15, 2014; the disclosures of which applications are herein incorporated by reference.

### INTRODUCTION

Mammalian body temperature is normally controlled by an internal autonomic regulatory system referred to herein as the thermoregulatory system. One important effector in this system is by controlled by blood flow to specialized skin areas of the body at non-hairy skin surfaces (i.e., at the palms, soles of the feet, cheeks/nose regions). Subcutaneous to these areas, there are unique anatomical vascular structures called venous plexuses. These structures serve to deliver large volumes of blood adjacent the skin surface. By this delivery of blood, significant heat transfer is enabled for the maintenance of internal organs within a functional temperature range. Blood is permitted to pass through the venous plexuses “radiator” structures by way of arterio venous anastomosis, or AVAs that gate or control the arterial input side of the venous plexuses. Thus, the AVA's serve an integral part of the heat transfer system, providing thermoregulatory control. Together, the AVA's and venous plexuses comprise a body's relevant heat exchange vasculature.

Normally, when body and or environmental temperatures are high, dilation of certain blood vessels favors high blood flow to the noted heat exchange surfaces, thus increasing heat loss to the environment and reduction in the deep body core region temperature. As environmental and/or body temperatures fall, vasoconstriction reduces blood flow to these surfaces and minimizes heat loss to the environment.

There are situations, however, in which it would be desirable to manipulate the transfer of heat across skin surfaces to modulate the core body temperature. Such core body cooling or heating would be useful in a number of applications, including therapeutic treatment regimens and as a component of improving athletic or industrial performance.

### SUMMARY

Systems and methods for transferring heat with a portion of a mammal are provided. Aspects of the subject systems include a negative pressure device configured to apply negative pressure to a portion of a mammal, and a temperature modulation device configured to contact the portion of the mammal under negative pressure in a manner sufficient to transfer heat with the contacted portion of the mammal, e.g., either remove heat or introduce heat into the contacted portion of the mammal. Also provided are kits that find use in practicing embodiments of the methods. The disclosed systems and methods find use in a variety of different applications, including both therapeutic and non-therapeutic applications.

### BRIEF DESCRIPTION OF THE FIGURES

FIG. 1 provides a partial cross-sectional top view of a system according to the subject embodiments.

FIG. 2 provides a rear perspective view of an embodiment of a negative pressure device that can be employed to practice to the subject methods.

FIGS. 3 to 5 provide multiple views of an embodiment of a cooling device that can be employed in the subject systems and to practice to the subject methods.

FIG. 6 provides a representation of an embodiment of a cooling device that can be employed in accordance with the embodiments of the systems and methods disclosed herein.

### DETAILED DESCRIPTION

Systems and methods for transferring heat with a portion of a mammal are provided. Aspects of the subject systems include a negative pressure device configured to apply negative pressure to a portion of a mammal, and a temperature modulation device configured to contact the portion of the mammal under negative pressure in a manner sufficient to transfer heat with the contacted portion of the mammal, e.g., either remove heat or introduce heat into the contacted portion of the mammal. Also provided are kits that find use in practicing embodiments of the methods. The disclosed systems and methods find use in a variety of different applications, including both therapeutic and non-therapeutic applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, subject systems and devices for use in practicing the subject methods will be discussed in greater detail, followed by a review of methods and particular applications.

### Systems

As summarized above, aspects of the invention include systems configured to transfer heat with, e.g., remove heat from or introduce heat into, a portion of a mammal. Embodiments of the systems include a negative pressure device and a temperature modulation device, e.g., cooling or heating device, which are distinct components that are configured to work together to transfer heat from a portion of a mammal. As the negative pressure and temperature modulation devices are distinct components, they are not integrated, such that they can be separated from each other without in any way altering the physical structure of each other. For example, the temperature modulation device may be configured to be placed in and removed from an interior portion of the negative pressure device, without in any way altering the physical structure of the negative pressure device. As the temperature modulation and negative pressure devices are non-integrated, they are configured to fully operate separately from each other for their intended purpose, e.g., to produce a negative pressure environment and contact a surface of a mammal with a reduced temperature surface, e.g., as described in greater detail below.

In further describing various aspects of the invention, the negative pressure and temperature modulation devices are now described separately in greater detail.

### Negative Pressure Devices

As summarized above, systems of the disclosed embodiments include a negative pressure device. Negative pressure devices are devices configured to produce and/or retain a negative pressure in a portion or region of the device. As used herein, the phrase “negative pressure” refers to a pressure lower than ambient pressure under the particular conditions in which the device is employed or the method is performed, e.g., 760 mmHg at sea level. The magnitude of the decrease in pressure from the ambient pressure to negative pressure is, in some instances, 20 mmHg or more, such as 30 mmHg or more, including 35 mmHg or more, where the magnitude of the decrease may be as great as 85 mmHg or more, but also may be 60 mmHg or less, such as 50 mmHg or less. When the method is performed at or about sea level, the negative pressure ranges in some instances from 740 mmHg to 675 mmHg, such as from 730 mmHg to 700 mmHg, including from 725 mmHg to 710 mmHg.

The phrase “negative pressure device”, as used herein, refers to a device configured for inducing and/or maintaining negative pressure, e.g., negative pressure within a specific enclosed area of the device, such as an area enclosed by a portion of a negative pressure device. Such a negative pressure device may be configured for applying negative pressure for a length of time, e.g., a length of time for effective employment of the systems, devices and/or methods described herein, e.g., a length of time sufficient to induce a lower core body temperature of a mammal.

The enclosed portion in which negative pressure is produced is configured or dimensioned to receive and/or produce a portion, e.g., limb or extremity thereof, head, etc., of a mammal. The terms “mammal” and “mammals” are used broadly herein to describe organisms which are within the class mammalia, including the orders carnivore (e.g., dogs and cats), rodentia (e.g., mice, guinea pigs, and rats), and primates (e.g., humans, chimpanzees, and monkeys). Mammals may be subjects or patients, such as human subjects or patients. The terms “human” or “humans” may include human subjects or patients of both genders and at any stage of development (i.e., fetal, neonates, infant, juvenile, adolescent, adult), where in certain embodiments the human subject is a juvenile, adolescent or adult. While the devices and methods described herein may be applied on a human subject, it is to be understood that the subject devices and methods may also be applied on other subjects (that is, in “non-human subjects”).

As noted above, negative pressure elements may be configured to produce, or hold and/or receive, an enclosed portion of a mammal. As used herein, the phrase “enclosed portion of a mammal” refers to a portion of a mammal, e.g., a wrist and/or hand, foot and/or ankle, head and/or neck, which lies partially or substantially within, such as between two or more portions, e.g., opposite interior portions of, or encapsulated within, a negative pressure element. An enclosed portion of a mammal may be a portion of a mammal that is contained within or surrounded by a negative pressure environment induced by the negative pressure element. An enclosed portion of a mammal may be, for example, one or more of a hand, and/or a foot, and/or an arm, and/or a leg, and/or a finger, and/or a head, and the like, or any combination, as well as portion, thereof. An enclosed portion of a mammal may include one or more portions of a mammal, e.g., a mammalian heat transfer surface, which is a surface of a mammal through which heat may transfer, with which a heat exchange surface of a heat transfer element, as described in greater detail below, is configured to transfer heat.

A heat exchange surface is a surface on and/or in the body of a mammal through which heat may transfer between the core body and the environment of the mammal. Heat exchange surfaces of interest with the subject systems, devices, and methods include those found in various regions of the mammal, e.g., extremities. For example, such portions may include one or more of the arms, legs, palms, soles, foreheads, temples, occipital regions, and the like, or any combination thereof.

In some embodiments, negative pressure devices include a sealed enclosure, such as an enclosure for receiving and containing therein a portion of a mammal. In various instances, negative pressure devices are configured to induce and/or maintain negative pressure within the sealed enclosure. The volume of the negative pressure enclosure of the device may vary, ranging in some instances from 5 cm³ to 40 cm³, such as from 10 cm³ to 30 cm³, including from 10 cm³ to 20 cm³. Such a volume may be substantially the same volume as or a slightly larger volume (e.g., greater than by 1 cm³ or less, 5 cm³ or less, 10 cm³ or less, 50 cm³ or less, or 100 cm³ or less) than the volume of a portion of a mammal enclosed within the negative pressure device. Such a volume may be sized to fit, e.g., to encapsulate, 95% of human hand sizes and/or 95% of human foot sizes. Alternately, it may be sized for more specific groups, such as children.

In some embodiments, the negative pressure devices are configured to be portable. For example, negative pressure devices may be sized and shaped to be easily moved from one location to another by an amount of force capable of being exerted by an average child and/or adult human arm and/or hand. In certain aspects, the negative pressure devices described herein have a mass ranging, for example, from 100 g to 3000 g, from 140 g to 2900 g, from 500 g to 2000 g, or from 1300 g to 1400 g.

Various embodiments of negative pressure devices include one or more openings (e.g., two, three, four, five openings) configured to receive a portion of a mammal, e.g., a portion of a mammal including a heat exchange surface. Such an opening may separate the interior form the exterior of the device and may be shaped as any convenient shape, e.g., a circle, oval, square, rectangle, triangle, or any combination thereof. An opening of a negative pressure device may also be sized to receive a portion of a mammal therethrough, e.g., a hand and/or a foot. An opening of a negative pressure device may have, for example, an area of 25 cm² or less, 100 cm² or less, or 225 cm² or less. The opening of a negative pressure device may be the only single opening from the interior to the exterior of the device. If there are a plurality of openings on the device, the openings may also be at opposite first and second ends of the device.

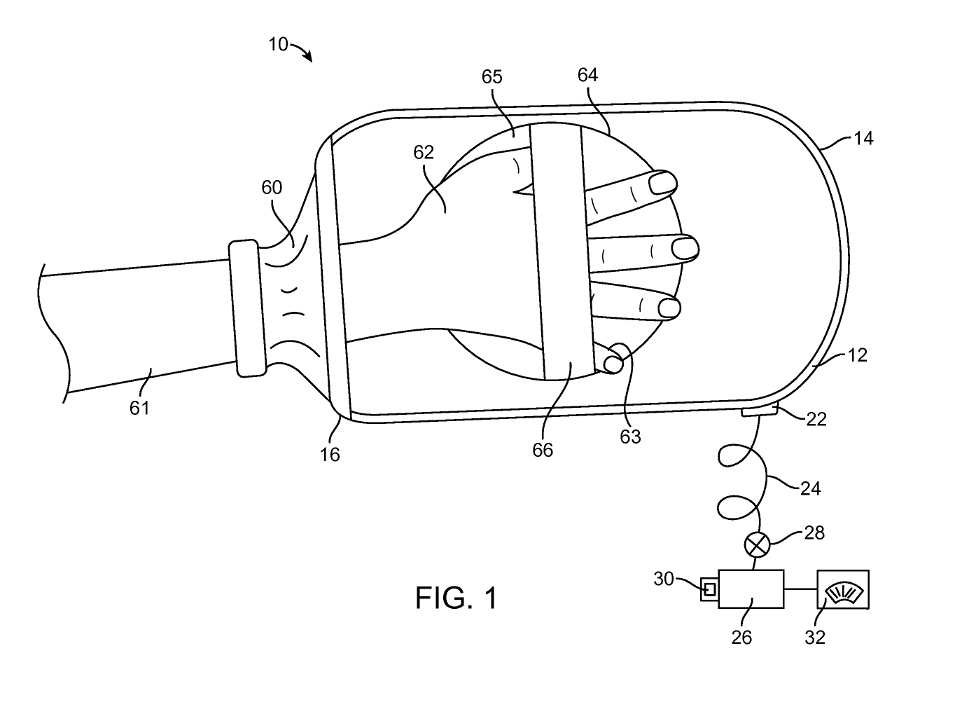

In some embodiments of negative pressure devices, the devices include one or more sealing elements (e.g., one, two, three, four, five sealing elements) configured to produce a seal, e.g., a reversible seal, about e.g., around, a portion of a mammal, e.g., a portion of a mammal received into and/or contained within a device. By “reversible seal” is meant a seal which can be repeatedly broken (e.g., made to not seal) and re-sealed. A seal may be an air-tight and/or water-tight seal and/or a seal between two different pressure levels, e.g., a negative pressure level and another pressure level, e.g., a pressure level immediately surrounding a negative pressure device. Such a seal may separate two pressure levels and maintain the difference between one pressure level and another. A sealing element of a negative pressure device may also form, e.g., be form-fitted, around a portion of a mammal, e.g., an arm and/or a wrist. A sealing element may be a portion of a negative pressure device which contacts a portion of a mammal. A sealing element may close an air and/or water-permeable opening between a negative pressure device and a portion of a mammal, e.g., such that the opening is made air-tight and/or water-tight. Sealing elements may be proximate (e.g., adjacent and/or attached to) an opening, or a plurality of openings, on a negative pressure device. One embodiment of a sealing element is shown, for example in FIG. 1 as sealing element 60, and is described in further detail below.

The seal may be a hard or soft seal. A “hard” seal is characterized as one designed to altogether avoid air leakage past the boundary it provides. In theory, a “hard” seal will allow a single evacuation of the negative pressure chamber for use in the methods. In practice, however, a “hard” seal can produce a tourniquet effect. Also, any inability to maintain a complete seal will be problematic in a system requiring as much. A “soft” seal as described herein is characterized as providing an approximate or imperfect seal at a user/seal interface. Such a seal may be more compliant in its interface with a user. Indeed, in response to user movement, such a seal may leak or pass some air at the user/seal interface. In a negative-pressure system designed for use with a soft seal, a regulator or another feedback mechanism/routine will cause a vacuum pump, generator, fan or any such other mechanism capable of drawing a vacuum to respond and evacuate such air as necessary to stabilize the pressure within the chamber, returning it to the desired level. Active control of vacuum pressure in real-time or at predetermined intervals in conjunction with a “soft” seal provides a significant advantage over a “hard” seal system that relies on simply pulling a vacuum with the hopes of maintaining the same.

One embodiment of a system including a negative pressure device for use in practicing the subject methods is provided in FIG. 1. The embodiment of the negative pressure device illustrated is a core body cooling apparatus 10. As shown in FIG. 1, the core body cooling apparatus 10 includes a sealed enclosure 12. Sealed enclosure 12 is dimensioned to fully or partially fit around, e.g., encapsulate, an enclosed portion of a mammal, e.g., a portion of an appendage, e.g., a hand 62.

Sealed enclosure 12 can be made of virtually any non-hazardous material or combination of materials/structures, which retains the requisite shape while the interior of sealed enclosure 12 is maintained at negative pressures, where in the broadest sense the material may be rigid or flexible (where in certain embodiments when the material is flexible, it is supported by a rigid framework). In some instances, sealed enclosure 12 is configured to support negative pressures down to −85 mmHg, or in certain embodiments, −85 mmHg or lower. In one embodiment, sealed enclosure 12 is flexible and may be made of pliant and elastic materials which can include supporting or reinforcing members. This type of construction easily accommodates movements of a hand 62 and thus provides the mammal with more comfort and freedom during practice of the subject methods than would a rigid enclosure. In some embodiments sealed enclosure 12 is a neoprene-impregnated polyester sheath supported on a spring steel wire helix.

Sealed enclosure 12, as shown in FIG. 1, has a distal end or rim 14 and a proximal rim 16. Distal rim 14 is, in some aspects, capped by a sealing element (not shown) capable of creating an airtight seal. In certain embodiments, sealed enclosure 12 may be closed off at distal end 14.

A sealing element 60 may, in some aspects, be flexible and/or be attached to proximal rim 16. Sealing element 60, in various embodiments, is made of a synthetic material impermeable to air, e.g., Neoprene®, or substantially impermeable to air. The tubular form of sealing element 60 ensures that it fits snugly around arm 61 and conforms to the arm's shape.

In the embodiment shown in FIG. 1, sealed enclosure 12 is provided with a pressure inlet 22. A pressure conduit 24, e.g., a flexible tube, is connected to inlet 22. The other end of conduit 24 is connected to a vacuum pump 26. In some embodiments, a pressure inlet may be connected directly to a vacuum pump. In some embodiments, a pressure inlet and/or a pressure conduit is absent from the device. Vacuum pump 26 is a standard pump capable of generating negative pressures, e.g., pressures down to −85 mmHg or, in some embodiments −85 mmHg or less, inside sealed enclosure 12. The delivery of this negative pressure through conduit 24 can be regulated by any conventional mechanisms. In some embodiments, a vacuum pump has an on/off switch 30 e.g., a manually operable on/off switch, configured to turn the vacuum pump on and/or off. In some embodiments, the on/off switch is connected to a timer which is configured to turn the switch on and/or off after a particular length of time. In some embodiments, the on/off switch is connected to a pressure gauge which and is configured to turn on and/or off, e.g., automatically turn on and/or off, when a particular pressure is reached within the device. In the embodiment shown, an adjustable valve 28 guarantees maintenance of the desired pressure inside sealed enclosure 12. Conveniently, a readout gauge 32 is also provided for visual pressure indication. In some variations, the battery and/or adjustable valve 28, and/or readout gauge 32, and/or conduit 24 and/or inlet 22 are not included on a negative pressure device.

In some embodiments, vacuum pump 26 is connected, e.g., electrically coupled, to a power source. A power source may, in some aspects, be a battery, e.g., a portable and/or self-contained battery, an outlet, or another source of electrical power. In some embodiments, the battery and/or the vacuum pump 26, and/or an adjustable valve 28, and/or readout gauge 32, and/or conduit 24 and/or inlet 22 are self-contained, e.g., attached and/or positioned in a portable and/or convenient manner, on a negative pressure device, e.g., on the exterior of the sealed enclosure 12 of the negative pressure device, e.g., a core body cooling apparatus 10. In some variations of the subject systems, the battery and/or the vacuum pump 26, and/or an adjustable valve 28, and/or readout gauge 32, and/or conduit 24 and/or inlet 22 are positioned within, or substantially within a negative pressure device. Accordingly, in some aspects, negative pressure devices, e.g., core body cooling apparatuses, include an integrated source of electrical power, e.g., a battery. As such, in some embodiments, the negative pressure devices are configured to be portable. For example, negative pressure devices may be sized and shaped to be easily moved from one location to another by an amount of force capable of being exerted by an average child and/or adult human arm and/or hand. While the embodiment depicted in FIG. 1 shows a separate, operably connected vacuum source, the vacuum source may be integrated into the device, and may or may not include the gauges, etc., as depicted.

As is described further in the methods section below, core body cooling apparatus 10 is simple to use. First, a mammal's hand 62, and in some embodiments, a temperature modulation, e.g., cooling, device 64, is inserted through sealing element 60 and thereby placed inside sealed enclosure 12. In this position, sealing element 60 wraps around a portion of arm 61. To ensure that sealing element 60 conforms closely to the contour of the portion of arm 61, the latter is bare.

With hand 62, and in some embodiments temperature modulation, e.g., cooling, device 64, properly inserted into sealed enclosure 12, pump 26 is activated to produce a negative pressure, e.g., a pressure between −20 mmHg and −85 mmHg, inside sealed enclosure 12. Sealing element 60, under the influence of negative pressure or suction, seals tightly around the upper part of arm 61 to preserve the vacuum inside sealed enclosure 12.

The negative pressure device shown in FIG. 1 and described below is merely one embodiment of devices that can be employed, e.g., within the disclosed systems, to practice the subject invention. Other negative pressure device configurations are possible, and come within the scope of the subject disclosure.

FIG. 1 also depicts one embodiment of a temperature modulation, e.g., cooling device 64 which can be utilized, e.g., within the subject systems, in accordance with the subject methods. The embodiment of the cooling device shown in FIG. 1 is described further below.

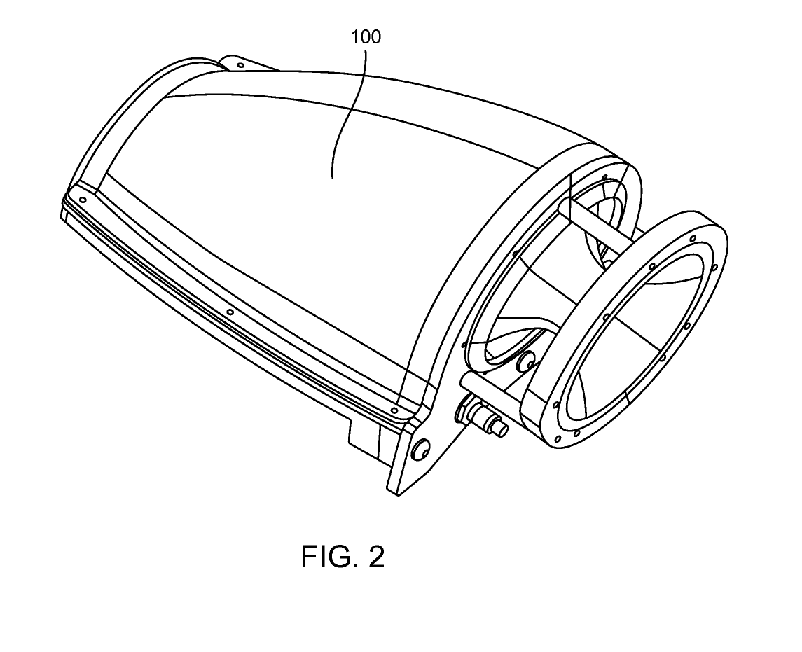

FIG. 2 provides a view of an embodiment of a negative pressure device 100 that can be employed to practice the subject invention. The embodiment of the negative pressure device shown in FIG. 2, and variations thereof, which may be used in accordance with the subject systems and methods, are described in greater detail in U.S. Pat. Nos. 6,656,208; 6,974,442; and 8,177,826, the disclosures of which are incorporated by reference herein. Also of interest are negative pressure devices as described in greater detail in U.S. Pat. Nos. 6,602,277; 7,182,776; 8,277,496; 6,637,099; 6,966,922; 7,862,600; 8,287,581; 7,122,047; and 7,947,068; as well as Published PCT Application WO/1996/028120; the disclosures of which applications are herein incorporated by reference.

The device for transferring heat shown in FIGS. 1 and 2 is merely one embodiment of devices that can be employed to practice the subject methods. Other device configurations are possible and come within the scope of the subject disclosure. For example, the enclosures may take the form of mittens, gloves, etc., e.g., for enclosing hands or portions thereof; shoes, boots, socks, etc., e.g., for enclosing feet or portions thereof; helmets, hats, caps, etc., e.g., for enclosing heads or portions thereof. For example, a helmet such as a football helmet, batting helmet, lacrosse helmet, hockey helmet, bicycle helmet, ski helmet, snow board helmet, etc., may be configured to include a heating element and negative pressure component, e.g., as described herein.

### Temperature Modulation Devices

Systems of the disclosed embodiments include one or more temperature modulation device, which may be heating or cooling devices. Heating devices are any devices that are configured to introduce heat to the enclosed portion of the mammal. As such, the heating devices are configured to cause energy transfer into a subject from the heating device upon contact of the heating device with a heat exchange surface of the subject. Embodiments of the subject cooling devices are configured to contact a portion of a mammal, such as a heat exchange surface and operate, either alone or with a negative pressure device, to remove heat from the mammal, for example, to lower the core body temperature of a mammal. As such, the cooling devices are configured to cause energy transfer from a subject to the cooling device upon contact of the cooling device with a heat exchange surface of the subject. As reviewed above, aspects of certain embodiments include the temperature modulation, e.g., cooling, devices as separate and detached from negative pressure devices of the disclosed systems.

In further describing this aspect of the invention, the temperature modulation device will be described primarily in terms of a cooling device. The description of the cooling device is readily applicable to heating device embodiments, where the cooling medium is exchanged for a heating medium.

Cooling devices may have any convenient size or shape to operate for the purposes described herein. For example a cooling device may have one or more surfaces for contacting a portion of a mammal, such as a heat exchange surface, e.g., a palm and/or sole. As such, a cooling device may be a sheet, such as a sheet configured to be wrapped around a portion of a mammal. A cooling device may also be a pouch having an opening for receiving a portion of a mammal therein. In certain aspects, cooling devices are shaped substantially like a glove or a mitten. In certain embodiments, cooling devices are substantially planar. Various embodiments of cooling devices have an outer periphery defining a shape which is, or substantially is, that of a circle, oval, rectangle (e.g., a rectangle having rounded corners), square, triangle, or any combination thereof. Certain variations of cooling devices have an outer periphery defining a single continuous edge. Cooling devices may also include one or more sealed or sealable receptacles (e.g., an openable and closable receptacle) for receiving into and/or sealing one or more substances within the cooling devices. Such receptacles may be actuable between open and closed positions. In some embodiments, cooling devices are configured, e.g., sized and/or shaped to fit entirely within, e.g., between at least two portions of, a negative pressure device while an enclosed portion of a mammal, e.g., an enclosed portion stably associated with the cooling device, is also within the negative pressure device.

Embodiments of cooling devices include one or more, e.g., one, or a plurality of, such as two, three, four, or more, containers, e.g., a sealed container. A container of a cooling device may include one compartment or a plurality, e.g., two, three, four, five, ten or fewer, fifty or fewer, one-hundred or fewer, such as between five and one-hundred, such as between forty and sixty, such as between forty-five and fifty-five, of compartments therein. Compartments of cooling device may be one or more pockets, e.g., sealed pockets, such as air and/or water-tight pockets, within the cooling device.

In certain embodiments, containers are flexible containers. For instance, a flexible container may be compliant, for example, such that it is configured to form a form-fitted surface which is complementary with a portion of a mammal, such as a heat exchange surface, against which it is placed. In various embodiments, containers, such as flexible containers, are polymeric containers. Polymeric containers are containers which have one or more portion composed of one or more polymeric materials. Polymeric containers may be entirely or partially composed of one or more polymeric materials, e.g., a single polymeric material. Specific polymeric materials of interest include, but are not limited to: plastics, rubbers, silicones, etc.

In various embodiments, containers are rigid containers. For instance, a rigid container may be non-compliant, or substantially non-compliant, for example, such that it does not change its shape when contacted with an aspect, such as a portion of a mammal, such as a heat exchange surface, against which it is placed. In various embodiments, containers, such as rigid containers, are polymeric containers.

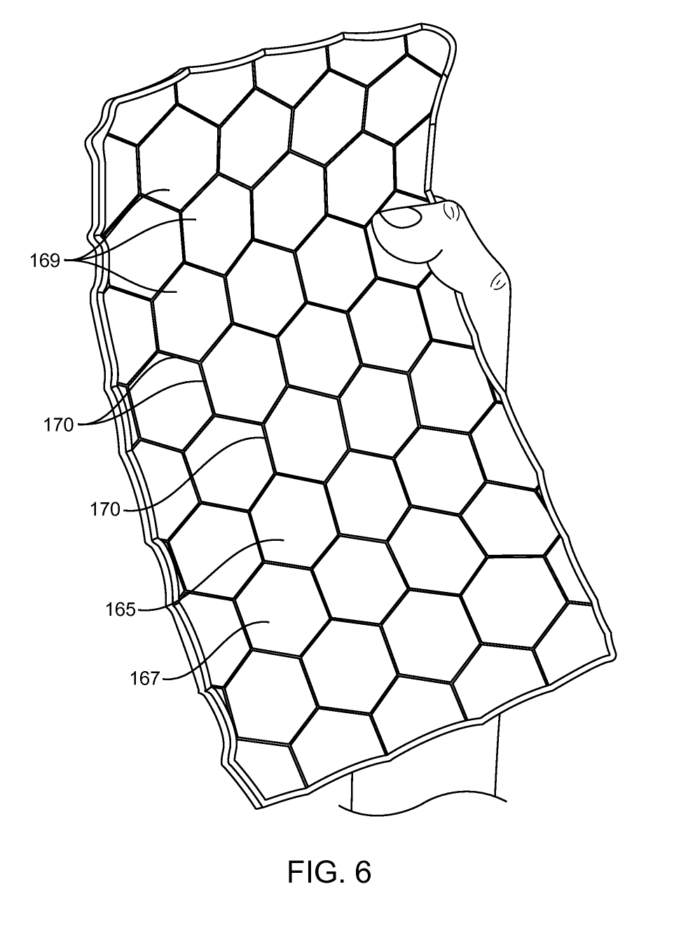

In embodiments of cooling devices including a plurality, such as two, three, four, or more, containers, the containers may be affixed to one another at edge portions of the containers. In such embodiments, the cooling devices may be configured such that the cooling device and portions thereof, e.g., the container, may flex despite each individual container or pocket thereof containing therein a solid material, e.g., a phase change material. As noted above, in some embodiments, containers include a network of pockets. Pockets of containers may have a peripheral shape that is or substantially is that of a rectangle, square, triangle, diamond, circle, oval, hexagon, octagon, or any combination thereof. Such pockets may be connected to each other by edge portions of containers. In some embodiments, including embodiments of containers having pockets shaped as hexagons, the pockets may be positioned for example, with respect to one another, in a honeycomb arrangement, e.g., joined to one another, for example at 6 or fewer sides. One embodiment of a container having a honeycomb arrangement is shown, for example, in FIG. 6. However, as noted above, any suitable shape may be employed for such pockets, including an octagonal shape e.g., joined to one another, for example at 8 or fewer sides and/or a triangular shape e.g., joined to one another, for example at 3 or fewer sides.

Certain aspects of cooling devices are disposable devices. In other words, the devices are configured for disposal following one or more uses, such as a single use. Embodiments of cooling devices and the components thereof, e.g., a container and/or an attachment element, are composed of one or more biodegradable, e.g., organic, materials.

Containers of cooling devices or portions thereof, such as compartments of cooling devices, in certain variations, contain (i.e., seal therein) a cooling medium. In the broadest sense, the cooling medium may be a material or composition that is capable of attaining a temperature that, upon contact of the cooling device with the heat exchange surface of a mammal, results in transfer of energy from the mammal to the cooling medium. The cooling medium may be a gas, liquid or solid, and may or may not include water.

In some instances, the cooling medium is a phase change material. By “phase change material” is meant a material which is configured to undergo a change, and in some instances repeated changes, from one phase to another, e.g., solid to liquid and/or liquid to solid, when exposed to mediums, e.g., air, a surface of a mammal, etc., at various temperatures. Various embodiments of phase change materials are configured to change phase from a solid to a liquid (i.e., melt) after a period of time following contact with a portion of a mammal, such as a heat exchange surface. Such a period of time may range from 0.1 min to 120 min, such as from 0.5 min to 60 min, including from 1 min to 10 min, such as from 1 min to 12 min, e.g., from 1 min to 30 min.

Certain embodiments of phase change materials have a first “initial” temperature at which the materials are maintained. Such an initial temperature may be a temperature at which the phase change materials are a solid, e.g., maintained entirely in a frozen state. An initial temperature of a phase change material may be achieved by, for example, placing the phase change material in a cooler, e.g., a household refrigerator or freezer, for a period of time necessary for the phase change material to freeze.

Phase change materials also have a temperature, e.g., a second temperature, and/or a subsequent temperature, at which the material, or at least a portion thereof, change from a solid to a liquid. In various embodiments, the temperature at which a phase change material is configured to change phase from a solid to a liquid may range, for example, from 1° C. to 35° C., from 10° C. to 30° C., from 10° C. to 25° C., from 10° C. to 22° C., or from 12° C. to 19° C. The second temperature of the phase change materials may occur following or be induced by a period of time of exposure or contact between the phase change material and a portion of a mammal, such as a heat exchange surface. Such contact or exposure may be through a portion of a container containing the phase change material which is positioned immediately adjacent to or contacting a portion of a mammal. A period of time of exposure or contact between the phase change material and a portion of a mammal may be a period of time required for energy, such as thermal energy, such as thermal energy from a portion of a mammal, to be absorbed by the phase change material. Additionally, in embodiments of phase change materials in liquid form, the materials assume the shape of the interior of the container of the cooling device in which the materials are contained.

Certain variations of phase change materials are configured to maintain the environment immediately surrounding (e.g., contacting, and/or within 1 cm, 2 cm, 5 cm, 10 cm, or 20 cm, or within a negative pressure device) the material at the same temperature, or substantially the same temperature (e.g., within 1° C., 2° C., or 5° C.), as that of the phase change material. Embodiments of phase change materials are configured to maintain the environment immediately surrounding the materials at a substantially constant (e.g., within 0.5° C., within 1° C. or within 2° C.) temperature of 15.6° C. for a length of time, e.g., 5 min, 10 min, 30 min, 1 hr, 2 hr, 6 hr, 24 hr, or a length of time required for the materials to fully melt. Certain embodiments of phase change materials are configured to maintain the environment immediately surrounding the materials at a substantially constant temperature after the phase change materials have been maintained in a solid state (i.e., frozen).

The temperature of phase change materials, e.g., phase change materials within a container of a cooling device, may vary, but in some aspects is not so low as to cause local vasoconstriction at a surface of the mammal, e.g. a heat exchange surface. The phase change materials may have a temperature, e.g., a first temperature or a second temperature, ranging from 1° C. to 35° C., from 10° C. to 30° C., from 10° C. to 25° C., from 10° C. to 22° C., or from 12° C. to 19° C. In certain embodiments, phase change materials have a temperature, e.g., a first temperature or a second temperature, which is a temperature that provides for thermal energy extraction from the core body of a mammal and not local vasoconstriction.

In various aspects, phase change materials are configured such that contact may be maintained between the materials and a mammal for a period of time sufficient for a desired amount of core body thermal energy extraction to occur. Such a period of time may be, for example, 1 min or less, 2 min or less, 3 min or less, or 5 min or less, and/or such contact may be maintained for 10 hr or less, such as 1 hour or less, such as 5 min or less.

In certain embodiments, phase change materials may include one or more oils e.g., a single oil or combination of 2 or more distinct oils. In embodiments of phase change materials which are oils, the materials may one or more synthetic oils or naturally occurring oils. Phase change materials may have a variety of colors (e.g., white, green, blue, grey, black, red, yellow, or any combinations thereof) and can be substantially opaque or transparent.

Any convenient phase change material (PCM) may be employed. PCMs of interest, but are not limited to: organic PCMs (including paraffins and non-paraffins), inorganic PCMs, and eutectics, etc. Examples of organic PCMs that may be used include, but are not limited to, n-tetradeca, formic acid, n-pentadeca, acetic acid, n-hexadeca, caprilone, docasyle, n-henicosan, phenol, n-lauric, p-joluidine, cynamide, n-docosane, n-tricosane, hydrocinna, cetyl, o-nitroanili, camphene, diphenyl, n-penta cosane, myristic acid, oxolate, tristearin, o-xylene, β-chloroacetic, n-hexacosane, nitro naphthalene, a-chloroacetic, n-octacosane, palmitic acid, bees wax, glyolic acid, p-bromophenol, azobenzene, acrylic acid, dintro toluene, phenylacetic acid, and/or thiosinamine. Examples of inorganic PCMs that may be used include but are not limited to water, POCl₃, SbCl₅, H₂SO₄, MOF₆, P₄O₆, H₃PO₄, Cs, Ga, AsBr₃, Bl₃, TiBr₄, H₄P₂O₆, SO₃, and/or SbCl₃. According to another aspect of the disclosure, the PCMs may be solid-solid PCMs. The PCMs store heat energy by transitioning from one crystalline structure to another. Such transitions have the advantage that in some cases they occur with less volume change in the material, and thus may be an appropriate choice for certain applications. Examples of solid-solid PCMs that may be used in embodiments of the invention include, but are not limited to, penterythritol, neopentyl glycol, trihydroxy mthyl-aminomethane, diamnopentacrythritol, trumethylolethane, pentaglycerin, monoaminopentaerythritol, tris(hydroxymethyl) acetic acid, and combinations of the aforementioned solid-solid PCMs.

Specific PCMs of interest include, but are not limited to, those sold by Phase Change Energy Solutions (www.phasechange.com) and PCM Products (www.pcmproducts.net).

In various embodiments, phase change materials are materials which are may be employed in cold-pack devices. Such cold-pack devices may be those utilized in health-related applications, e.g., to reduce swelling of a portion of a mammal. Such cold-pack devices may also be those utilized in food-related applications, e.g., to retain food in a cool environment which discourages growth of bacteria.

Embodiments of subject cooling devices include one or more, e.g., one, such as only a single one, or a plurality, such as two, three, four, or more, attachment elements. Such an attachment element may be configured to secure a container of a cooling device to a portion of a mammal, e.g., a heat exchange surface. In securing a container to a portion of a mammal, the attachment element may cause the container to be retained in a position immediately adjacent to and/or contacting and/or pressing against, the portion of the mammal for the duration of use of the cooling device or longer.

In various embodiments, an attachment element, or portion thereof, e.g., a strap, is configured to secure a container to a portion of a mammal, e.g., a hand or foot. In various embodiments, attachment elements, or portions thereof, e.g., straps, are configured to secure a container to a portion of a mammal by forming an opening between the attachment element and the container. Such an opening may be formed on a first side by the attachment element and formed on a second side which is opposite the first side by the container. Such an opening may also be configured to receive, e.g., slidably receive, a portion of a mammal, e.g., a portion of a hand or foot, therethrough. Additionally, an opening in a cooling device may be sized and shaped such that a fit, e.g., a friction fit, e.g., a friction fit which is comfortable to the mammal, is obtained between the portion of the mammal and the cooling device, wherein the fit may affix the portion of the mammal to the cooling device. An opening formed between an attachment element and a container may be sized and/or shaped to have a minimal impact, e.g., no impact, on the circulation of blood through a portion of the mammal inserted therein and/or therethrough. Furthermore, an attachment element, such as an attachment element composed of an elastic material, may be biased to retain a portion of a mammal, e.g., a hand or foot, between an attachment element and a container, e.g., within an opening in a cooling device. An attachment element may also be biased to retain, e.g., retain by a friction fit, a portion of a mammal within, e.g., between two or more portions of, a cooling device.

Attachment elements, in various aspects, include one or more, e.g., two, three, four, etc., straps. Straps may have any suitable size and/or shape. For example, a strap may be substantially planar and have a peripheral shape defining or substantially defining a rectangle, square, oval, or other shape. In certain embodiments, straps are rectangular and have a length, width and height. In various aspects, the length and width of straps are both many times e.g., 5 or more, 10 or more, 20 or more, or 50 or more times, longer than the height of a strap.

In some embodiments, straps have a first end and a second end and are affixed at the first and second end to the container of a cooling device. In some aspects, a first end of a strap is affixed to or proximate a first edge of a container and/or a second end of a strap is affixed to or proximate a second edge of a container, and/or wherein the second edge of a container is on an opposite side of the container than the first edge. In certain aspects, attachment elements, e.g., straps, are attached to a container fixedly, e.g., by adhesive, and/or by a sewn seam, and/or by a melted seam. In some embodiments, attachment elements are a continuous body, e.g., body of material, with and/or composed of the same material as a container or a portion of a container of a cooling device. In various embodiments, portions of attachment elements, such as straps, e.g., first and second ends, protrude into portions of a container, e.g., portions of a container proximate (e.g., within 1 cm, or, 2 cm, or 5 cm of) one or more locations where the attachment elements are affixed to the container.

In certain embodiments, attachment elements, such as straps, have more than one portion, e.g., two portions, which are removably and repeatedly attachable to one another. Such portions may be attachable to one another by a connection formed, for example, by Velcro™, and/or one or more snaps, and/or buttons, and/or holes, and/or hooks, and/or clips, such as clips or buckles, e.g., plastic or metal clips or buckles, having a reciprocating male and female portion.

Attachment elements, such as straps, or portions thereof, may be composed of any suitable material. For example, attachment elements may be composed of one or more fabrics, e.g., cotton and/or polyester and/or nylon. Attachment elements may also be entirely or partially composed of one or more elastic materials. Elastic materials are materials which, for example, are biased to retain their initial un-biased shape when one or more forces are exerted thereon which cause the materials to assume, e.g., to temporarily assume, a second “biased” shape. Various aspects of attachment elements, such as attachment elements having elastic materials are configured to temporarily assume a second “biased” shape when a portion of a mammal is inserted into and/or retained within a cooling device, such as between a container and an attachment element. Embodiments of elastic materials are materials which are configured to exert, when retained in a “biased” shape, a force, a sufficient force, on a portion of a mammal and/or one or more portions of a container such that the surface area between the container and a portion of a mammal, such as a heat exchange surface, is maximized. Furthermore, attachment elements or portions thereof, e.g., straps, and/or elastic materials of attachment elements, may be composed of one or more polymeric materials, e.g., a single polymeric material. Specific polymeric materials of interest include, but are not limited to: plastics, rubbers, silicones, etc.

Materials of which attachment elements may be composed also include materials which have the requisite strength for the disclosed systems, devices and methods to effectively be employed. For example, materials of which attachment elements are composed include materials which are resistant to degrading and/or tearing and/or breaking when attachment elements are repeatedly employed to secure a container to a portion of a mammal, such as a heat exchange surface. Materials of which attachment elements may be composed also include materials which are sterilizable and/or re-sterilizable. Materials of which attachment elements are composed, in various embodiments, also include materials which are disposable, e.g., disposable after a single use, such as biodegradable and/or organic materials.

In various aspects of cooling devices, the devices include one or more pads, such as a pad which is attached or detached from the remaining portions of the cooling device e.g., the container and/or the attachment element. Aspects of pads of the disclosed cooling devices may be configured to enhance a subject mammal's comfort, e.g., comfort relating touch and/or temperature, during employment of the subject systems, device and methods. Various aspects of pads of the disclosed cooling devices are configured to increase the effectiveness of the subject systems, device and methods, for example, by assisting to maintain a temperature of or immediately surrounding a phase change material within a container during operation of the disclosed systems and devices. Embodiments of pads of cooling devices are sized and/or shaped to fit, e.g., comfortably fit, against and/or around a portion of a mammal, e.g., a palm. Certain embodiments of pads of cooling devices are sized and/or shaped to fit within a negative pressure device while other portions of a cooling device, e.g., a container and/or an attachment element, are retained therein as well.

FIG. 1 depicts one embodiment of a cooling device 64 which can be utilized, e.g., within the subject systems, in accordance with the subject methods. More specifically, FIG. 1 shows a cooling device 64 in use with, and positioned entirely within, an embodiment of a negative pressure device, depicted as core body cooling apparatus 10. FIG. 1 illustrates the cooling device 64 stably associated with a heat exchange surface 63 of an enclosed portion of a mammal. The phrase “stably associated” is described further below. FIG. 1 specifically depicts several portions of the cooling device 64 including container 65, and attachment element 66. As shown in FIG.1, the portion of the mammal including the heat exchange surface 63 is inserted into an opening in the cooling device between the container 65 and the attachment element 66. It is notable that cooling device 64 may also be configured to be employed, for example, with any of the embodiments of the negative pressure device described herein.

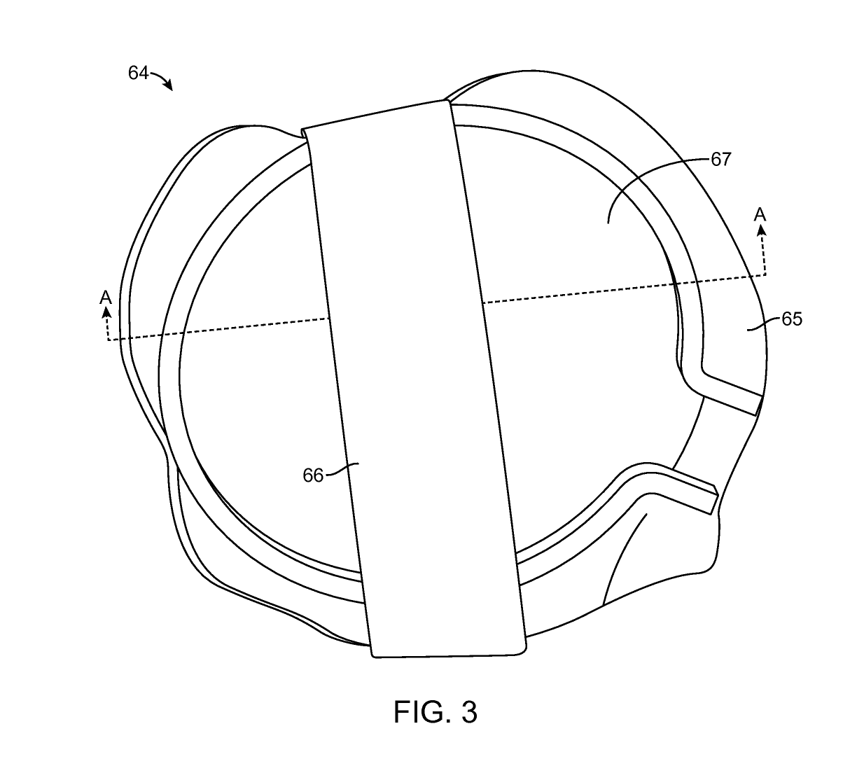

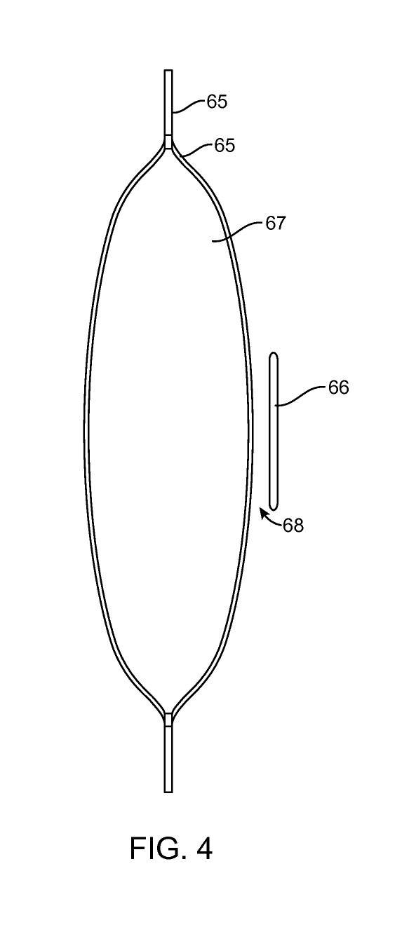

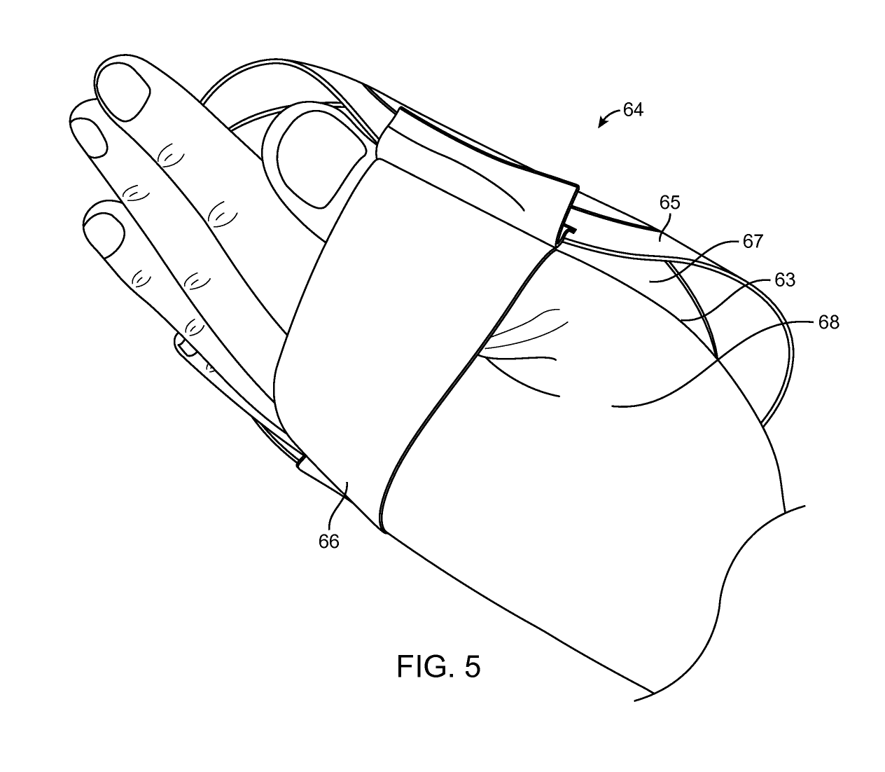

One embodiment of a cooling device 64 is shown, for example, in FIGS. 3 to 5. The embodiment of the cooling device 64 depicted in FIGS. 3 to 5 may be employed, for example, with the embodiment of the negative pressure device shown in FIG. 1 according to the methods described herein to lower the core body temperature of a mammal. FIG. 3 specifically provides a top-down view of one embodiment of the cooling device 64 whereas FIG. 4 provides a cross sectional view of the cooling device 64 shown in FIG. 3, taken along the cross sectional plane designated A-A. Likewise, FIG. 5 provides a perspective view of the embodiment of the cooling device 64. FIGS. 3 to 5 illustrate various embodiments of elements of the cooling device 64 shown including a container 65 and an attachment element 66. The attachment element 66 is shown in FIGS. 3 and 5 connected to the container 65, e.g., fixedly connected to the container 65 at a first location and a second location. FIG. 5 also depicts phase change material 67 shown contained within the container 65. FIGS. 3 and 5 illustrate phase change material 67, e.g., a white, opaque, solid and/or liquid phase change material, shown through at least partially transparent container 65 of the cooling device 64. FIG. 4 additionally depicts one embodiment of an opening 68, as described further above, which may be configured to receive a portion of a mammal, e.g., a portion of a mammal including a heat exchange surface, therein and/or therethrough. Likewise, FIG. 5 shows a portion of a mammal, e.g., a portion of a mammal 68, such as a hand, including a heat exchange surface 63, inserted into and/or stably associated with cooling device 64 between attachment element 66 and container 65.

Another embodiment of a cooling device 164 is shown in FIG. 6. FIG. 6 illustrates an embodiment of a cooling device 164 having a container 165 including plurality of pockets 169, e.g., individually sealed pockets. In the embodiment shown, the plurality of pockets 169 are separated by individually sealed edges 170 and have a hexagonal shape. In addition, phase change material 167, e.g., an at least partially transparent, solid and/or liquid phase change material, is shown through at least partially transparent container 165 of the cooling device 164. The systems and devices shown in the figures may be employed in accordance with any of the subject methods described below.

While the devices disclosed above include devices where the heat transfer element includes a phase change material, embodiments of interest also include those having heat transfer elements that do not include phase change materials. For example, the devices may include thermoelectric elements as heat transfer elements. Of interest are thermoelectric heating elements, e.g., that may include a resistive conductors, Peltier devices, or other element that generates heat. Examples of thermoelectric heating elements of interest include, but are not limited to, those described in U.S. Pat. Nos. 8,567,861; 7,663,378; 7,500,536; 7,202,444; 6,150,642; 5,894,207; 5,111,025; 4,825,048 and 4,700,046; the disclosure of the heating elements thereof being incorporated herein by reference.

Also of interest are thermoelectric cooling elements, e.g., that may include a Peltier device or other element that provides a cooled surface or component. Examples of thermoelectric cooling elements of interest include, but are not limited to, those described in U.S. Pat. Nos. 8,269,098; 6,894,215; 6,722,139; 6,574,967; 6,020,671; 5,950,067; 5,921,087; and 5,456,164; the disclosure of the cooling elements thereof being incorporated herein by reference. In such embodiments, the heat transfer element may include a material, e.g., a pad or analogous compliant structure, configured to be positioned between the heat transfer element and skin of the subject during use of the device, e.g., to improve comfort of the patient and/or decrease tissue damage to the patient.

### Methods

As described above, the subject disclosure provides methods for removing heat from a portion of a mammal e.g., the body core of a mammal. By body core is meant the internal body region or portion of the mammal, as opposed to the surface of the mammal. In various aspects, the methods include removing heat from a specific portion of a mammal, e.g., a hand and/or a foot.

In certain embodiments, the methods include stably associating a heat exchange surface of a portion of a mammal, with a cooling device. By “stably associating” and/or “stably associated” is meant placing in a contacting orientation, e.g., a contacting orientation wherein the surface area of the touching portions are maximized, and/or affixing to, e.g., affixing to with an attachment element, such that the disclosed methods, systems, and devices may be effectively employed. Heat exchange surfaces of interest with the subject methods include those found in various regions of the mammal, e.g., extremities. For example, heat exchange surfaces may include one or more of the arms, legs, palms, soles, and the like, or any combination thereof.

Cooling devices which may be employed with the subject methods include each of the cooling device embodiments described herein. For example, in some instances, cooling devices include a container, e.g., a flexible container, e.g., a polymeric container, containing a phase change material, e.g., an oil, e.g., a synthetic oil. In some embodiments of the methods, a phase change material is configured to change phase from a solid to a liquid after a period of time following or upon contact with a heat exchange surface. Such a period of time may range from 0.1 min to 120 min, from 0.5 min to 60 min, from 1 min to 10 min, from 1 min to 12 min, or from 1 min to 30 min. In addition, in certain embodiments of the subject methods, a portion of a mammal is stably associated with a cooling device for a period of time ranging, for example, from 0.1 min to 120 min, from 0.5 min to 60 min, from 1 min to 10 min, from 1 min to 12 min, or from 1 min to 30 min. In various embodiments, the temperature at which a phase change material which is utilized with the subject methods is configured to change phase from a solid to a liquid may range, for example, from 1° C. to 35° C., from 10° C. to 30° C., from 10° C. to 25° C., from 10° C. to 22° C., or from 12° C. to 19° C.

In some instances, cooling devices utilized in accordance with the subject methods include one or more attachment elements configured to secure the container to a heat exchange surface of a portion of a mammal. In various embodiments of the methods, attachment elements are configured to stably associate a cooling device with a portion of a mammal, e.g., a heat exchange surface. The attachment elements employed with the subject methods may be any of the attachment elements described herein and may, for example, include a strap, and/or be configured to secure a container of a cooling device to a portion of a mammal, e.g., a hand and/or foot. In aspects of attachment elements used in accordance with the subject methods, the attachment element includes a strap which, in turn, includes one or more elastic material.

Various embodiments of the subject methods include removing a cooling device from a cold storage device. Removing a cooling device from a cold storage device may be performed, for example, prior to stably associating a portion of a mammal, e.g., a heat exchange surface, with a cooling device. A cold storage device is, in various embodiments, a device which maintains an environment therein which is sufficient to effectively perform the subject methods. An environment within a cold storage device may, in some embodiments, be colder than the environment immediately surrounding the cold storage device. In various aspects of cold storage devices, the temperature within the cold storage device is such that a phase change material of a cooling device is maintained in a solid state therein.

Embodiments of cold storage devices include refrigerators and/or freezers of a conventional commercial, industrial, and/or residential type. For example, a cold storage device may be a typical household freezer and/or refrigerator which may be configured for typical household freezer and/or refrigerator use such as storing food and/or drink. Such a cold storage device may be configured to maintain an environment, including a temperature, therein which is typical of a refrigerator and/or freezer of a conventional commercial, industrial, and/or residential type. Cold storage devices, in various embodiments, are configured to maintain a temperature, e.g., a temperature in one or more internal compartments thereof, in a temperature range, for example, from 1° C. to 35° C., from 10° C. to 30° C., from 10° C. to 25° C., from 10° C. to 22° C., or from 12° C. to 19° C.

In some embodiments, removing a cooling device, e.g., a cooling device entirely retained with a cold storage device, from a cold storage device includes grabbing the cooling device within the cold storage device and thereafter moving the cooling device to a location entirely outside the cold storage device. Removing a cooling device from, a cold storage device may also, for example, include causing the phase change material to change, or begin to change, from a solid to a liquid phase.

Some embodiments of the subject methods include placing a cooling device within a cold storage device. Placing a cooling device within a cold storage device may be performed, for example, after applying negative pressure to an enclosed portion of a mammal and/or removing heat from an enclosed portion of a mammal using a cooling device and/or a negative pressure device. Placing a cooling device within a cold storage device may also be performed prior to any of the other steps of the methods described herein. Placing a cooling device within a cold storage device may include causing the phase change material to change, or begin to change, from a liquid to a solid phase.

In certain embodiments, the methods include placing a portion of a mammal, e.g., a portion of a mammal that is stably associated with a cooling device, into a negative pressure device, e.g., a negative pressure device configured to receive and enclose the portion of a mammal, such as any of the negative pressure devices described herein, and apply negative pressure thereto. Placing a portion of a mammal stably associated with a cooling device into a negative pressure device may include inserting both the portion of the mammal and the cooling device into the negative pressure device and/or retaining the portion of the mammal and the cooling device therein.

In various aspects of the methods, the methods include applying negative pressure to, or inducing negative pressure conditions on, a portion of a mammal, e.g., a portion of a mammal enclosed within a negative pressure device. In various embodiments, applying negative pressure to a portion of a mammal, e.g., a portion of a mammal enclosed within a negative pressure device, removes heat from, i.e., cools, the portion of the mammal, e.g., the enclosed portion.

In practicing the subject methods, negative pressure conditions may be provided using any convenient protocol. In certain embodiments, applying negative pressure is achieved, and/or negative pressure conditions are provided, by enclosing a portion of a mammal, e.g., a portion of a mammal that includes a target surface that is to be contacted with a low temperature medium, e.g., a cooling device, in a sealed enclosure, e.g., a sealed enclosure of a negative pressure device, where the pressure is then reduced in the sealed enclosure thereby providing the requisite negative pressure conditions. The portion that is enclosed in the sealed enclosure is a portion of the mammal that includes, for example, the target heat exchange surface, and therefore is an appendage in some embodiments of the subject invention. As such, the portion that is sealed is an arm or leg, or at least a portion thereof, e.g. hand or foot, in various embodiments of the subject invention.

In some embodiments, negative pressure devices applied in accordance with the disclosed methods provide a sealed enclosure, such as enclosure for receiving and containing therein a portion of a mammal. Negative pressure devices may be configured to induce and/or maintain negative pressure within the sealed enclosure. A sealed enclosure of a negative pressure device may, in various aspects, have a volume ranging, for example, from 5 cm³ to 40 cm³, such as from 10 cm³ to 30 cm³, or from 10 cm³ to 20 cm³. Such a volume may be substantially the same volume as or a slightly larger (e.g., greater than by 1 cm³ or less, 5 cm³ or less, 10 cm³ or less, 50 cm³ or less, or 100 cm³ or less) volume than the volume of a portion of a mammal enclosed within the negative pressure device. A volume of a sealed enclosure may be sized to fit, e.g., to encapsulate, 95% of human hand sizes.

Additionally, in certain embodiments of negative pressure devices employed in accordance with the subject methods, the devices include one or more sealing elements (e.g., one, two, three, four, five sealing elements) configured to produce a seal, e.g., a reversible seal, about e.g., around, a portion of a mammal, e.g., a portion of a mammal received into and/or contained within a device. Furthermore, the subject methods may include making the subject systems and devices according to a suitable method.

In various embodiments of the methods, the mammal is a female, e.g., a female human, or a male, e.g., a male human. In some aspects of the methods wherein the mammal is female or a male, the methods are methods of treating the female or male for a hot flash. By treating a female or male for a hot flash is meant operating to alleviate a hot flash, e.g., a hot flash caused by changing hormone levels, e.g., reduced levels of estrogen, such as changing hormone levels that are characteristic of menopause. The term “hot flash” may refer to standard hot flashes and/or “slow hot flashes” or “ember flashes”. In various aspects, hot flashes according to the subject methods include an elevated body temperature, e.g., an elevated core body temperature. In some aspects, hot flashes according to the subject methods include a perceived elevated body temperature.

In certain embodiments, the subject disclosure provides methods for enhancing the ability of mammal to perform a physical procedure. By enhancing is meant improving or bettering the ability of the mammal to perform a particular physical procedure, task or operation.

In practicing the subject methods, thermal energy is extracted from the body core of the mammal at least once during a hot flash or during physical activity to result in the desired treatment of the hot flash or ability enhancement. By “body core” and “core body” is meant the internal region of the mammal, as opposed to the surface of the mammal. The magnitude of core body thermal energy extraction accomplished during practice of the subject methods may vary, and is sufficient to provide for the desired outcome, e.g. reduction in core body temperature, treatment of a hot flash, ability enhancement, relief from hyperthermia, or MS symptoms, etc., and the like. In certain embodiments, the magnitude of heat extraction is 0.5 Kcal/min or less, 1.0 Kcal/min or less, or 15Kcal/min or less, where the magnitude may be 50 Kcal/min or greater, but sometimes is 30Kcal/min or less, such as 20 Kcal/min or less. The period of time that the heat is extracted from the core body may range, for example, from 1 min to 24 hrs, such as from 2 min to 20 min, or from 2 min to 5 min.

In certain embodiments, the core body temperature of the subject is reduced. The magnitude of core body temperature reduction is sufficient to provide for treatment of a hot flash or ability enhancement, and is, in some aspects, 0.5° C. or less, or 1.0° C. or greater, or 1.5° C. or greater, or may be 4° C. or greater, or 4.0° C. or less, or 2.0° C. or less. The period of time that the core body temperature is reduced may range from 1 min to continuous for the duration of a hot flash or activity. For example, the period of time that the core body temperature is reduced may range from 2 min to 20 min, or from 2 min to 5 min. In some embodiments, the subject methods will prevent or minimize rises in the core body temperature. Nonetheless, in these embodiments the subject methods do extract heat or thermal energy from the core body of the subject, but the amount of energy being produced by or introduced into the core body of the subject from other sources is substantially the same as or exceeds the amount of energy being extracted from the core body by the subject methods.

Where the specific embodiment is a method of treating a hot flash or enhancing physical ability, the heat or thermal energy is extracted from the core body at least once during the hot flash or physical procedure, where the hot flash or procedure is measure from a point prior to the beginning of the hot flash or procedure to the end of the hot flash or procedure, e.g., to the end of a period of an actual and/or perceived raised body temperature associated with a hot flash, to the end of a training set, to the end of a game, to the end of given work day, etc. In certain embodiments, core body heat is extracted a plurality of times. Where core body heat is extracted a plurality of times, the number of different times that heat is extracted may, for example, range from 2 to 20, such as from 2 to 15, such as from 5 to 10. In certain embodiments, core body thermal energy is extracted a single time. The term procedure is used broadly to include anything from a single physical movement to a plurality of physical movements that are practiced in a given period of time, e.g. participation in a game, performing a particular training regimen, activity encountered during an entire workday etc.

In extracting core body thermal energy from the mammal, a surface of the mammal may be contacted and/or stably associated with a low temperature medium, such as a cooling device, under negative pressure conditions for a period of time sufficient to achieve the desired reduction in core body temperature. The phrase “negative pressure conditions”, as used herein, refers to conditions under negative pressure, as negative pressure is described herein. The surface that is contacted and/or stably associated with the low temperature medium is, in some aspects, a heat exchange surface. Heat exchange surfaces of interest with the subject methods include those found in various regions of the mammal, e.g. the arms, legs, palms, soles, head, face, ears, and the like.

As described above, the surface of the mammal is contacted with a low temperature medium under the negative pressure conditions. By low temperature medium is meant a medium, such as a cooling device, that has a temperature that is sufficient to provide the requisite core body thermal energy or heat extraction or removal. The temperature of the low temperature medium may vary, but, in some aspects is not so low as to cause local vasoconstriction at the surface of the mammal, e.g. the heat exchange surface. The low temperature medium, in some aspects, has a temperature ranging, for example, from 1° C. to 35° C., from 10° C. to 30° C., from 10° C. to 25° C., from 10° C. to 22° C., or from 12° C. to 19° C. In certain embodiments, a feature of the subject methods is that the temperature of the low temperature medium is specifically selected to be one that provides for thermal energy extraction from the core body and not local vasoconstriction. Contact and/or stable association may be maintained for a period of time sufficient for the desired amount of core body thermal energy extraction to occur. As such, contact may be maintained for 1 min or more, such as 2 min or more, or 3 min or more, where contact may be maintained for 10 hr or longer. In some embodiments, contact is maintained for 1 hr or less, or 5 min or less.

In practicing the subject methods, the negative pressure conditions during contact and/or stable association may be static/constant or variable. Thus, in certain embodiments, a negative pressure is maintained at a constant value during contact of a portion of a mammal, e.g., a heat exchange surface, with the low temperature medium, e.g., a cooling device. In yet other embodiments, the negative pressure value is varied during such contact, e.g. oscillated. Where the negative pressure is varied or oscillated, the magnitude of the pressure change during a given period may be varied and may range from −85 mmHg to 40 mmHg, such as from −40 mmHg to 0 mmHg. In addition, in some aspects, the periodicity of the oscillation may range from 0.25 sec to 10 min, such as from 1 sec to 5 min, or from 1 sec to 10 sec.

In certain embodiments, the subject methods further include a feedback element that at least partially controls when a heat exchange surface of the mammal is contacted and/or stably associated with a low temperature medium, e.g., a cooling device, to extract thermal energy from the core body of the mammal. The feedback element may be any convenient element, where a suitable element is a thermosensor, e.g. placed over a heat exchange surface not being contacted with a low temperature medium, e.g., a cooling device. In such embodiments, the method, in some aspects, further includes a data processing step for processing the feedback data and activating the contact and/or stable association with the low temperature medium in response thereto, e.g. a computing element that controls the contact of the heat exchange surface with the low temperature medium. Additionally, as noted above, the subject methods are suitable for use with a variety of mammals.

### Utility

As demonstrated above, the subject methods are directed to transferring, e.g., removing or introducing, heat from the body core of a mammal. As such, the subject methods are suitable for use in a variety of different applications, where particular applications include the treatment of normal and abnormal physiological conditions, e.g., disease and/or discomfort, where core body heat extraction is desirable. Particular applications in which the subject methods find use include the alleviation or treatment of hot flashes, treatment of exercise or work induced hyperthermia, treatment of stroke, treatment of cystic fibrosis symptoms, treatment of multiple sclerosis symptoms, and the like. By treatment is meant at least an alleviation in one or more of the symptoms associated with the condition being treated, e.g. a reduction in discomfort, amelioration or elimination of symptoms, etc.

In various embodiments, the subject systems and methods are applied for alleviation or treatment of hot flashes. For example, a mammal undergoing a hot flash and/or a perceived hot flash may utilize the negative pressure devices with the cooling devices described herein to create a drop in core body temperature of the mammal and thereby provide relief from the hot flash. Relief and/or treatment of a hot flash provided by the subject systems, devices and methods may be related to making a mammal, e.g., a mammal experiencing a hot flash, more comfortable, such as less scared or agitated. Relief and/or treatment of a hot flash may also be that related to alleviating a health risk, e.g., a significant health risk, to a mammal associated with a hot flash.

In some aspects, a mammal undergoing a hot flash may stably associate a cooling device with a heat exchange surface of the mammal and thereafter insert the cooling device with a portion of the mammal including the heat exchange surface into a negative pressure device. The mammal may then operate the negative pressure device to lower the core body temperature of the mammal and thereby alleviate the hot flash. In some aspects, mammals may utilize the systems and devices described herein in a preemptive manner. For example, a mammal expecting to undergo a hot flash may utilize the subject systems, devices and methods to forestall or prevent a hot flash.

In certain embodiments, the subject methods are employed for enhancing the ability of a mammal to perform a physical procedure or task, e.g., an athletic and/or work related physical procedure or task. As such, the subject methods are suitable for use in a variety of different applications where a variety of different types of physical procedures are performed. Examples of devices and methods which may be used either wholly or partially in connection with the disclosed systems, devices and methods to, for example, enhance the ability of a mammal to perform a physical procedure or task, or for another use, are provided by U.S. Pat. Nos. 6,656,208; 6,974,442; and 8,177,826, the disclosures of which are incorporated by reference herein. The methods and devices as described herein also find use in the applications as described in greater detail in U.S. Pat. Nos. 6,602,277; 7,182,776; 8,277,496; 6,637,099; 6,966,922; 7,862,600; 8,287,581; 7,122,047; and 7,947,068; as well as Published PCT Application WO/1996/028120; the disclosures of which are herein incorporated by reference.

### Kits

Also provided are kits that at least include the subject systems and devices and which may be used according to the subject methods. The subject kits may include two or more, e.g., a plurality, three, four, five, ten, etc., temperature modulation, e.g., cooling, devices according to any of the embodiments described herein, or any combinations thereof. In addition, the kits may include any device or other element which may facilitate the operation of any aspect of the kits. For example, a kit may include one or more cold storage device and/or one or more pads of a cooling device. Kits may also include packaging, e.g., packaging for shipping the systems and/or devices without breaking.

In certain embodiments, the kits which are disclosed herein include instructions, such as instructions for using devices. The instructions for using devices are, in some aspects, recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the systems or devices or as a website address with which instructions posted on the world wide web may be accessed.

Notwithstanding the appended clauses, the disclosure is also defined by the following clauses:
1. A system comprising:

(a) a negative pressure device configured to produce an enclosed portion of a mammal and apply negative pressure to the enclosed portion; and

(b) a temperature modulation device that is separate from the negative pressure device and comprises:
(i) a container comprising a temperature modulating medium; and
(ii) an attachment element configured to secure the container to a heat exchange surface of a portion of a mammal.
2. The system according to Clause 1, wherein the enclosed portion of the mammal is a hand or foot.
3. The system according to Clause 2, wherein the enclosed portion is a hand.
4. The system according to any of Clauses 1 to 3, wherein the heat exchange surface is a palm or sole.
5. The system according to any of Clauses 1 to 4, wherein the container is a flexible container.
6. The system according to any of Clauses 1 to 4, wherein the container is a rigid container.
7. The system according to any of Clauses 1 to 6, wherein the temperature modulation device is a cooling device comprising a cooling medium.
8. The system according to Clause 7, wherein the cooling medium comprises a phase change material.
9. The system according to Clause 8, wherein the phase change material is configured to change phase from a solid to liquid after a period of time following contact with the heat exchange surface.
10. The system according to Clause 9, wherein the period of time ranges from 0.5 to 60 minutes.
11. The system according to Clause 8, wherein the phase change material is configured to change phase from a solid to a liquid when it reaches a temperature ranging from 10° C. to 25° C.
12. The system according to Clause 8, wherein the phase change material comprises an oil.
13. The system according to Clause 12, wherein the oil comprises a synthetic oil.
14. The system according to any of Clauses 1 to 13, wherein the attachment element comprises a strap configured to secure the container to a hand or foot.
15. The system according to Clause 14, wherein the strap comprises an elastic material.
16. The system according to any of Clauses 1 to 15, wherein the negative pressure device is configured to provide a sealed enclosure having a volume ranging from 10 cm³ to 20 cm³.
17. The system according to Clause 16, wherein the negative pressure device comprises an opening configured to receive portion of a mammal and a sealing element configured to produce a reversible seal about the received portion.
18. The system according to any of Clauses 1 to 17, wherein the negative pressure device has a mass ranging from 100 g to 3000 g.
19. A method of removing heat from a portion of a mammal, the method comprising:

(a) stably associating a heat exchange surface of a portion of a mammal with a cooling device, wherein the cooling device comprises:
(i) a container comprising a cooling medium; and
(ii) an attachment element configured to secure the container to a heat exchange surface of a portion of a mammal to stably associate the cooling device with the heat exchange surface;

(b) placing the portion of the mammal that is stably associated with the cooling device into a negative pressure device configured to receive and enclose the portion of a mammal and apply negative pressure thereto; and

(c) applying negative pressure to the enclosed portion of the mammal to remove heat from the portion of the enclosed portion of the mammal.
20. The method according to Clause 19, wherein the portion of the mammal is a hand or foot.
21. The method according to Clause 20, wherein the portion is a hand.
22. The method according to any of Clauses 19 to 21, wherein the heat exchange surface is a palm or sole.
23. The method according to Clause 22, wherein the heat exchange surface is a palm.
24. The method according to any of Clauses 19 to 23, wherein the container is a flexible container.
25. The method according to any of Clauses 19 to 23, wherein the container is a rigid container.
26. The method according to any of Clauses 19 to 25, wherein the cooling medium comprises a phase change material.
27. The method according to Clause 26, wherein the phase change material is configured to change phase from a solid to liquid after a period of time following contact with the heat exchange surface.
28. The method according to Clause 27, wherein the period of time ranges from 0.5 to 60 minutes.
29. The method according to Clause 28, wherein the portion is stably associated with the cooling device for a period of time ranging from 0.5 to 60 minutes.
30. The method according to any of Clauses 26 to 29, wherein the phase change material is configured to change phase from a solid to a liquid when it reaches a temperature ranging from 10° C. to 25° C.
31. The method according to any of Clauses 26 to 30, wherein the phase change comprises an oil.
32. The method according to Clause 31, wherein the oil comprises a synthetic oil.
33. The method according to any of Clauses 19 to 32, wherein the attachment element comprises a strap configured to secure the container to a hand or foot.
34. The method according to Clause 33, wherein the strap comprises an elastic material.
35. The method according to any of Clauses 19 to 34, wherein the negative pressure device is configured to provide a sealed enclosure having a volume ranging from 10 cm³ to 20 cm³.
36. The method according to Clause 35, wherein the negative pressure device comprises an opening configured to receive portion of a mammal and a sealing element configured to produce a reversible seal about the received portion.
37. The method according to any of Clauses 19 to 36, wherein the negative pressure device has a mass ranging from 100 g to 3000 g.
38. The method according to any of Clauses 19 to 37, wherein the method further comprises removing the cooling device from a cold storage device.
39. The method according to any of Clauses 19 to 37, wherein the mammal is a female.
40. The method according to Clause 39, wherein the method is a method of treating the female for a hot flash.
41. A cooling device configured to remove heat from a portion of a mammal, the cooling device comprising:

(i) a container comprising a phase change material; and

(ii) an attachment element configured to secure the container to a heat exchange surface of the portion of a mammal.
42. The cooling device according to Clause 41, wherein the portion of the mammal is a hand or foot.
43. The cooling device according to Clause 42, wherein the portion is a hand.
44. The cooling device according to Clause 41, wherein the heat exchange surface is a palm or sole.
45. The cooling device according to Clause 44, wherein the heat exchange surface is a palm.
46. The cooling device according to any of Clauses 41 to 45, wherein the container is a flexible container.
47. The cooling device according to Clause 46, wherein the flexible container is a polymeric container.
48. The cooling device according to any of Clauses 41 to 45, wherein the container is a rigid container.
49. The cooling device according to any of Clauses 41 to 48, wherein the phase change material is configured to change phase from a solid to liquid after a period of time following contact with the heat exchange surface.
50. The cooling device according to Clause 49, wherein the period of time ranges from 0.5 to 60 minutes.
51. The cooling device according to any of Clauses 41 to 50, wherein the phase change material is configured to change phase from a solid to a liquid when it reaches a temperature ranging from 10° C. to 25° C.
52. The cooling device according to any of Clauses 41 to 51, wherein the phase change material comprises an oil.
53. The cooling device according to Clause 52, wherein the oil comprises a synthetic oil.
54. The cooling device according to any of Clauses 41 to 53, wherein the attachment element comprises a strap configured to secure the container to a hand or foot.
55. The cooling device according to Clause 54, wherein the strap comprises an elastic material.
56. A kit comprising two or more cooling devices according to any of Clauses 41 to 55.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A system comprising:
(a) a negative pressure device configured to produce an enclosed portion of a mammal and apply negative pressure to the enclosed portion; and
(b) a temperature modulation device that is separate from the negative pressure device and comprises:
(i) a container comprising a temperature modulating medium; and
(ii) an attachment element configured to secure the container to a heat exchange surface of a portion of a mammal.

2. The system according to claim 1, wherein the enclosed portion of the mammal is a hand or foot.

3. The system according to claim 1 or 2, wherein the temperature modulation device is a cooling device comprising a cooling medium.

4. The system according to claim 3, wherein the cooling medium comprises a phase change material.

5. The system according to claim 4, wherein the phase change material is configured to change phase from a solid to liquid after a period of time following contact with the heat exchange surface.

6. The system according to claim 5, wherein the period of time ranges from 0.5 to 60 minutes.

7. The system according to claims 4 to 6, wherein the phase change material is configured to change phase from a solid to a liquid when it reaches a temperature ranging from 10° C. to 25° C.

8. The system according to claim 7, wherein the phase change material comprises an oil.

9. The system according to claim 8, wherein the oil comprises a synthetic oil.

10. The system according to any of claims 1 to 9, wherein the attachment element comprises a strap configured to secure the container to a hand or foot.

11. The system according to any of claims 1 to 10, wherein the negative pressure device is configured to provide a sealed enclosure having a volume ranging from 10 cm³ to 20 cm³.

12. The system according to claim 11, wherein the negative pressure device comprises an opening configured to receive portion of a mammal and a sealing element configured to produce a reversible seal about the received portion.

13. The system according to any of claims 1 to 12, wherein the negative pressure device has a mass ranging from 100 g to 3000 g.

14. A method of removing heat from a portion of a mammal, the method comprising:
(a) stably associating a heat exchange surface of a portion of a mammal with a cooling device of a system according to any of claims 1 to 13;
(b) placing the portion of the mammal that is stably associated with the cooling device into a negative pressure device of a system according to any of claims 1 to 13; and
(c) applying negative pressure to the enclosed portion of the mammal to remove heat from the portion of the enclosed portion of the mammal.

15. A cooling device configured to remove heat from a portion of a mammal, the cooling device comprising:
(i) a container comprising a phase change material; and
(ii) an attachment element configured to secure the container to a heat exchange surface of the portion of a mammal.

What is claimed is:

1. A system comprising:
(a) a negative pressure device configured to produce an enclosed portion of a mammal and apply negative pressure to the enclosed portion; and
(b) a temperature modulation device that is separate from the negative pressure device and comprises:
(i) a container comprising a temperature modulating medium; and
(ii) an attachment element configured to secure the container to a heat exchange surface of a portion of a mammal.

2. The system according to claim 1, wherein the enclosed portion of the mammal is a hand or foot.

3. The system according to claim 1 or 2, wherein the temperature modulation device is a cooling device comprising a cooling medium.

4. The system according to claim 3, wherein the cooling medium comprises a phase change material.

5. The system according to claim 4, wherein the phase change material is configured to change phase from a solid to liquid after a period of time following contact with the heat exchange surface.

6. The system according to claim 5, wherein the period of time ranges from 0.5 to 60 minutes.

7. The system according to claims 4 to 6, wherein the phase change material is configured to change phase from a solid to a liquid when it reaches a temperature ranging from 10° C. to 25° C.

8. The system according to claim 7, wherein the phase change material comprises an oil.

9. The system according to claim 8, wherein the oil comprises a synthetic oil.

10. The system according to any of claims 1 to 9, wherein the attachment element comprises a strap configured to secure the container to a hand or foot.

11. The system according to any of claims 1 to 10, wherein the negative pressure device is configured to provide a sealed enclosure having a volume ranging from 10 cm³ to 20 cm³.

12. The system according to claim 11, wherein the negative pressure device comprises an opening configured to receive portion of a mammal and a sealing element configured to produce a reversible seal about the received portion.

13. The system according to any of claims 1 to 12, wherein the negative pressure device has a mass ranging from 100 g to 3000 g.

14. A method of removing heat from a portion of a mammal, the method comprising:
(a) stably associating a heat exchange surface of a portion of a mammal with a cooling device of a system according to any of claims 1 to 13;
(b) placing the portion of the mammal that is stably associated with the cooling device into a negative pressure device of a system according to any of claims 1 to 13; and
(c) applying negative pressure to the enclosed portion of the mammal to remove heat from the portion of the enclosed portion of the mammal.

15. A cooling device configured to remove heat from a portion of a mammal, the cooling device comprising:
(i) a container comprising a phase change material; and
(ii) an attachment element configured to secure the container to a heat exchange surface of the portion of a mammal.
